(19) United States
(12) Patent Application Publication
(10) Pub. No.: US 2022/0015673 A1
(43) Pub. Date: Jan. 20, 2022

(54) INTEGRATED OPTICAL BIOSENSORS INCLUDING MOLDED BEAM SHAPING ELEMENTS

(71) Applicant: ams AG, Premstaetten (AT)

(72) Inventor: Bernhard Gruber, Eindhoven (NL)

(21) Appl. No.: 17/414,837

(22) PCT Filed: Dec. 16, 2019
(86) PCT No.: PCT/EP2019/085460

§ 371 Date: Jun. 16, 2021

Related U.S. Application Data
(60) Provisional application No. 62/780,697, filed on Dec. 17, 2018.

(51) Int. Cl. A61B 5/1455; G02B 27/09

(52) U.S. Cl. CPC A61B 5/14552; G02B 1/041; A61B 2562/0238; G02B 27/0955

(57) ABSTRACT

An integrated optical biosensor module includes one or more light sources operable to produce light for emission from the module, and an integrated circuit chip including a photosensitive region. The photosensitive region includes one or more photodetectors operable to detect light produced by the one or more light sources and reflected by a subject that is outside the module. The integrated circuit chip is operable to determine a physiological condition of the subject based on signals from the one or more photodetectors. A clear mold covering encapsulates the one or more light sources, wherein the clear mold covering includes one or beam shaping elements each of which is disposed so as to intersect a path of a light beam from an associated one of the one or more light source.

### FIELD OF THE DISCLOSURE

The present disclosure relates to integrated optical biosensors that include one or more molded beam shaping elements.

### BACKGROUND

Various types of sensors are used in a wide range of applications. Some of these sensors use optical signals to measure parameters of interest, e.g., pressure, distance, temperature or composition. In some cases, modules incorporating such sensors are used in medical and health-related applications. For example, performing a measurement on a human body can include bringing a portion of the human body into proximity with the module, directing light emitted from the module toward the portion of the human body, and detecting light reflected by the portion of the human body into the module. Information based on the light detected by the module can be processed, for example, to provide an indication of a physiological condition of the human body.

### SUMMARY

The present disclosure describes integrated optical biosensors that include one more molded beam shaping elements (e.g., lenses). As described in greater detail below, the beam shaping element(s) can be formed integrally as part of a clear mold covering that encapsulates one or more light sources in the biosensor module.

For example, in one aspect, the present disclosure describes an integrated optical biosensor module that includes one or more light sources operable to produce light for emission from the module, and an integrated circuit chip including a photosensitive region. The photosensitive region includes one or more photodetectors operable to detect light produced by the one or more light sources and reflected by a subject that is outside the module. The integrated circuit chip is operable to determine a physiological condition of the subject based on signals from the one or more photodetectors. A clear mold covering encapsulates the one or more light sources, wherein the clear mold covering includes one or beam shaping elements each of which is disposed so as to intersect a path of a light beam from an associated one of the one or more light source.

Some applications include one or more of the following features. For example, each of the one or more beam shaping elements can be a molded lens composed of the same material as the clear mold covering. In some instances, the clear mold covering and the one or more beam shaping elements are composed of an epoxy resin.

In some implementations, the clear mold covering includes multiple beam shaping elements each of which is disposed so as to intersect a path of a light beam from a respective one of the light sources. At least one of the beam shaping elements may be asymmetric with respect to an optical axis of the light beam produced by the respective light source. In some cases, each of the respective beam shaping elements is operable to direct a light beam from a respective one of the light sources in a respective direction that differs from a direction in which a light beam from at least another one of the light sources is directed by a different one of the beam shaping elements.

In some implementations, each of the light sources is operable to produce light of a different wavelength, wherein the module includes multiple photodetectors each of which is operable to detect light produced by a respective one of the light sources and reflected by the subject. In some case, at least one of the light sources is operable to produce infra-red light or visible light. The clear mold covering should be transparent to the light produced by the one or more light sources.

In some cases, the module includes a housing defining an interior region in which the one or more light sources and the integrated circuit chip are disposed. The housing can have, for example, a first aperture over the clear mold covering, as well as a second aperture over the integrated circuit chip.

The module can be configured for use in various bio-sensor applications. For example, in some implementations, the integrated circuit chip is operable to determine an oxygen saturation level of the subject based on the signals from the one or more photodetectors. In some cases, the integrated circuit chip is operable to determine an pulse rate of the subject based on the signals from the one or more photodetectors. In some instances, the integrated circuit chip is operable to determine a heart rate of the subject based on the signals from the one or more photodetectors.

The disclosure also describes a host computing device that includes a cover glass and an integrated optical biosensor module disposed adjacent the cover glass. An application executable on the host computing device is operable to cause the module to perform a physiological measurement on the subject based on light produced by the one or more light sources, reflected by the subject, and sensed by the one or more photodetectors. The host computing device includes a display screen operable to display data indicative of the physiological condition of the subject based on the signals from the one or more photodetectors.

The disclosure further describes a system including an integrated optical biosensor module. The module includes one or more light sources operable to produce light for emission from the module, and an integrated circuit chip including a photosensitive region. The photosensitive region includes one or more photodetectors operable to detect light produced by the one or more light sources and reflected by a subject that is outside the module. A clear mold covering encapsulates the one or more light sources and includes one or beam shaping elements, each of which is disposed so as to intersect a path of a light beam from an associated one of the one or more light sources. The system also includes a processor coupled to the integrated circuit chip and operable to determine a physiological condition of the subject based on signals from the one or more photodetectors.

Some implementations include one or more of the following advantages. For example, the module can be relatively compact such that it can integrated into a host computing device (e.g., a smartphone or wearable device), in which space is at a premium. By integrating lenses or other beam shaping elements into the biosensor module, the energy of each light source can be focused more on the area to be measured, which can allow the aperture (and sensitivity) of the light source to be increased. Such arrangements can, in some cases, reduce power consumption of the overall system, which in turn can increase the lifetime of a battery that provides power to the biosensor module. Also, in some instances, beam shaping elements can reduce the crosstalk between the light source and the sensitive element and, therefore, improve overall system performance.

Forming the lenses or other beam shaping elements of the same epoxy resin such that they are integrated as part of the clear mold coverings over the light sources can allow a wide range of beam shaping elements to be provided, including asymmetrical lenses. Such arrangements can allow the light beams from the light sources to be directed in desired directions and to be optimized for a particular application.

Other aspects, features and advantages will be readily apparent from the following detailed description, the accompanying drawings, and the claims.

### BRIEF DESCRIPTION OF THE DRAWINGS

FIG. 1 illustrates a perspective view of an example of an integrated optical biosensor module.

FIG. 2 shows a cross-section of FIG. 1,

FIG. 3 illustrates a functional diagram and layout of an integrated circuit (IC) chip in the biosensor module.

FIG. 4 shows an example of a clear mold covering that includes beam shaping elements.

FIG. 5 shows an example of angular directions of beams emitted by the biosensor module.

FIG. 6 is another perspective view of the biosensor module.

FIG. 7 illustrates use of the biosensor module to obtain physiological data about a subject.

FIG. 8 illustrates an example of a portable computing device that includes the biosensor module.

### DETAILED DESCRIPTION

The present disclosure describes integrated optical biosensors that include one more molded beam shaping elements. As described in greater detail below, the beam shaping element(s) can be formed integrally as part of a clear mold covering that encapsulates one or more light sources. In some implementations, the techniques described here can provide greater flexibility in the arrangement of the beam shaping elements.

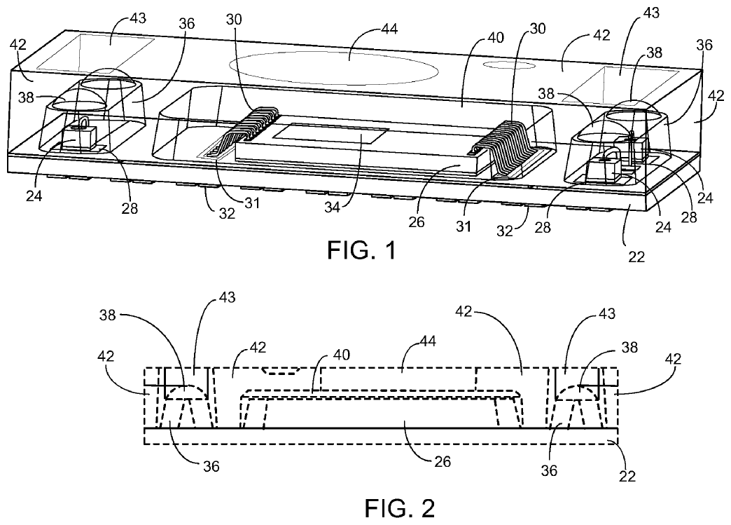

As shown, for example, in FIGS. 1 and 2, a packaged biosensor module 20 includes various components mounted to a printed circuit board (PCB) or other substrate 22. In particular, one or more light sources 24 and an integrated circuit (IC) semiconductor chip 26 are mounted to the PCB 22. The light sources 24 can be implemented, for example, as light emitting diodes (LEDs), organic LEDs (OLEDs), vertical cavity surface emitting lasers (VCSELs) or other light emitting devices. Each light source 24 is operable to produce light at a particular wavelength. In some instances, multiple light sources are mounted to the PCB 22, with each light source producing light of a different wavelength than one or more of the other light sources. For example, in some implementations, one light source is operable to emit light in the red part of the spectrum (e.g., about 600 nm), whereas a second light source is operable to emit light in the infra-red (IR) or near-IR part of the spectrum (e.g., in the range of about 700-1100 nm). Additional or different wavelengths or ranges of wavelengths may be used in other implementations. Each light source 24 can be connected to the PCB 22, for example, by way of a respective die pad 28. Likewise, the IC chip 26 can be connected electrically to the PCB 22, for example, by way of a die pad (not shown) and/or wirebonds 30 connected to pads 31 on the surface of the PCB 22. Surface mount technology (SMT) pads other electrical connections can be provided on the backside of the PCB 22 to facilitate electrical connection of the module 20 in a host device (e.g., a smartphone, wearable device, or other portable computing device).

The IC chip 26 has a photosensitive region 34 that includes one or more photodetectors (e.g., photodiodes), as well as circuity for controlling the light sources 24 and for processing signals from the photodetectors. The photodetectors are operable to sense the wavelength(s) of light produced by the light source(s) 24. If there are multiple light sources, each of which produces a different respective wavelength of light (e.g., in the IR or visible parts of the spectrum), then each photodetector can be configured (e.g., by the addition of an appropriate optical filter) to sense a different one of the wavelengths.

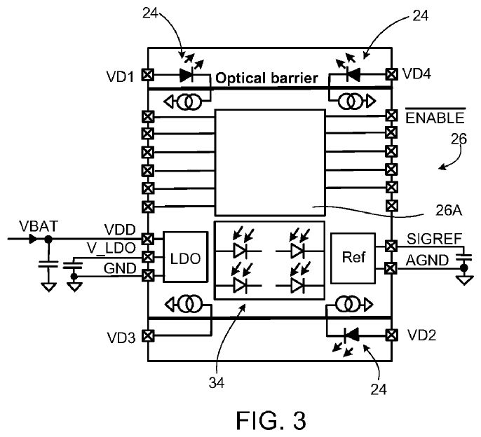

FIG. 3 illustrates a functional diagram and layout of the IC chip 26 according to a particular implementation. The IC chip 26A includes circuitry 26A for processing the photodiode output signals. The circuitry 26A can include, for example, optical front-end processing circuitry (e.g., synchronous demodulators; programmable sequencer; filter), electrical front-end processing circuitry (e.g., low noise analog circuitry), as well as circuitry to process the signals so as to generate an indication of a physiological condition of a living being (e.g., a person) based on signals from the photodiodes. The IC chip 26 can include various input/output pins and power supply connections. The IC chip 26 can store software instructions to implement the appropriate processing of the signals from the photodiodes. Various details may differ for other implementations.

As shown in FIGS. 1 and 2, the light sources 24 are encapsulated by a protective clear mold covering 36. In some instances, each light source 24 can be encapsulated by its own clear mold covering, whereas in other instances (e.g., as shown in the example of FIGS. 1 and 2), two or more light sources may be encapsulated by the same clear mold covering 36. The IC chip 26 also can be encapsulated by a protective clear mold covering 40. The clear mold coverings 36, 40, which can be formed by a molding process, can be composed, for example, of an epoxy resin that is substantially transparent to the wavelength(s) of light produced by the light source(s) 24.

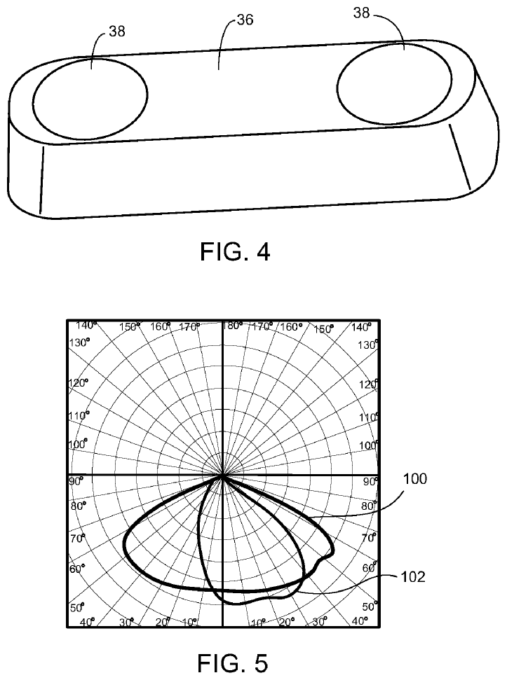

As further shown in FIGS. 1 and 2, as well as FIG. 4, the clear mold coverings 36 include respective beam shaping elements 38 that also can be formed during the molding process. Each light source 24 can have a respective beam shaping element 38 disposed so as to intersect the path of the light beam produced by the particular light source. Each beam shaping element 38 can shape (e.g., narrow or widen) the light beam produced by the associated light source 24. The beam shaping elements 38 can have a wide range of shapes. For example, the beam shaping elements 38 can include convex lenses or concave lenses. In some cases, Fresnel lenses can be provided. Further, the beam shaping elements 38 can differ from one another. By providing the lenses, the energy of each light source 24 can be focused more on the area to be measured, which can allow the aperture (and sensitivity) of the light source to be increased. This arrangement can, in some cases, reduce power consumption of the overall system, which in turn can increase the lifetime of a battery that provides power to the module 20.

Forming the beam shaping elements 38 of the same epoxy resin during the molding process so that they are integrated as part of the clear mold coverings 36 can allow a wide range of beam shaping elements to be provided. For example, in some instances, as illustrated in FIG. 4, a first one of the beam shaping elements 38A may be symmetrical with respect to the optical axis of the beam produced by the associated light source, whereas another one of the beam shaping elements 38B may be asymmetrical with respect to the optical axis of the beam produced by the associated light source. Such an arrangement can allow the light beams from the light sources 24 to be directed in desired directions and to be optimized for a particular application.

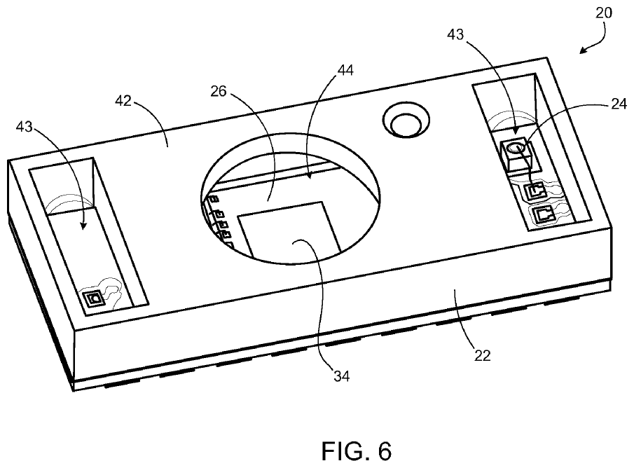

As an example, a biosensor module can include green, red and IR LEDs placed at the same distance from the sensor's photodiodes as one another. In the absence of lenses to direct the light beams in the desired directions, the longer wavelengths of the red and IR LEDs—compared to the wavelength of the green LED—typically would require that they be placed at a greater distance from the sensor. Providing asymmetric lenses over the red and IR lenses can deflect the beams into the desired areas of a target to be measured (a person's skin). See FIG. 5, which shows an example of the different angular spread of a first beam 100 and a second beam 102. Use of asymmetrical lenses can be used to allow a longer wavelength light source to be disposed on the PCB 22 relatively close to the associated photodetector, while the associated beam shaping element 38 for that light source is arranged so that the light beam produced by the light source is directed away from the photodetector. This arrangement can result in a highly compact module. Further, the lenses for the red and IR LEDs can have different optimal shapes according to their wavelengths and depending on the optical stack of the implementation, where the optical stack includes an air gap between the surface of the packaged biosensor and a cover glass of the host device), as well as depending on a thickness of the cover glass. The present techniques not only can achieve very compact packages, but also can help reduce crosstalk and improve signal-to-noise ratio. Such features also can allow larger apertures to be used over the sensor's photodiodes for a given overall module size.

As further shown in FIGS. 1, 2 and 6, in some implementations an opaque housing 42 is attached to the side of the PCB 22 on which the light sources 24 and IC 26 are mounted. The various components (e.g., the light sources 24, the IC chip 26 including the photosensitive region 34, and the clear mold coverings 36, 40 including the integrated lenses 38) are disposed in an interior region defined by the housing 42. The housing can be composed, for example, of a black epoxy or other polymer that is substantially non-transparent to the wavelengths of light emitted by the light sources 24 and that can be sensed by the photodetectors. The housing 42 includes apertures 43 above the clear mold coverings 36 over the light sources 24. Light produced by one of the light sources 24 passes through the associated lens 38 and exits the module via the associated aperture 43. Likewise, the housing 42 includes an aperture 44 above the clear mold covering 40 over the IC chip 26. Light reflected, for example, by human tissue can pass into the module 20 via the aperture 44 to be sensed by the photodetectors in the photosensitive region 34.

As noted above, the IC chip 26 is configured to generate an indication of a physiological condition of a living being (e.g., a person) based on signals sensed by the photodiodes. In operation, performing a measurement on a human body, for example, can include bringing a portion of the human body (a finger) into proximity with the module, directing light emitted from the module toward the portion of the human body, and detecting light reflected by the portion of the human body into the module. Information based on the light detected by the module can be processed by the IC chip 26, for example, to provide an indication of a physiological condition of the human body.

The IC chip 26 (or a processor in a host device in which the module is disposed) is configured to process the signals from the photodetectors in the integrated biosensor module in accordance with a particular application. In general, such applications include, but are not limited to, pulse oximetry, heart rate monitoring and photo-plethysmogram (PPG) applications.

Pulse oximeters, for example, are medical devices commonly used in the healthcare industry to measure the oxygen saturation levels in the blood non-invasively. A pulse oximeter can indicate the percent oxygen saturation and the pulse rate of the user. Pulse oximeters can be used for many different reasons. For example, a pulse oximeter can be used to monitor an individual's pulse rate during physical exercise. An individual with a respiratory condition or a patient recovering from an illness or surgery can wear a pulse oximeter during exercise in accordance with a physician's recommendations for physical activity. Individuals also can use a pulse oximeter to monitor oxygen saturation levels to ensure adequate oxygenation, for example, during flights or during high-altitude exercising.

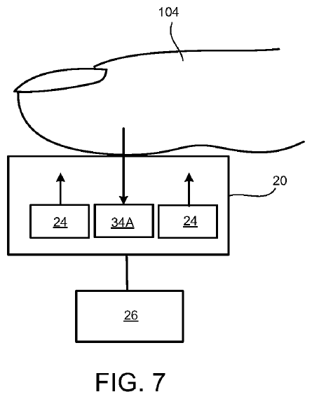

As illustrated by FIG. 7, during pulse oximetry applications, the subject (e.g., a person's finger 104) is illuminated by LEDs or other light sources 24 with light having two different wavelengths (e.g., infrared and visible red). As the light passes into the subcutaneous region and is incident on an arterial vessel, the oxygen-rich hemoglobin in the blood absorbs more of the light having the first wavelength and the hemoglobin without oxygen absorbs more of the light having the second wavelength. After absorption, the light is collected by one or more photodetectors 34A sensitive to the wavelengths of interest. The IC chip 26 (or another processor (e.g., microprocessor) in a host device) then determines the differences in absorption and converts the difference into information representative of the amount of oxygen being carried in the blood. The computation of the oxygen content may be performed according to any suitable algorithm known in the art.

For heart rate monitoring applications, the module 20 can be configured to emit light that illuminates the skin of a subject. A portion of the light passes through the skin into the subcutaneous tissue where it may encounter blood vessels carrying oxygenated arterial blood. With each cardiac cycle, the heart pumps blood through such vessels, causing the blood vessels to expand. The expansion and contraction of the blood vessels and the variation in the amount of oxygenated hemoglobin with each cycle modulates the light reaching the photodetectors in the module. By monitoring the time-varying change in the amount of light reflected back to and sensed by the module 20, the IC chip (or another processor (e.g., microprocessor) in a host device) can calculate the corresponding heart rate of the subject. The computation of the heart rate may be performed according to any suitable algorithm known in the art.

In some implementations, the module 20 is operable for PPG applications, which can use differential optical absorption spectroscopy (DOAS) techniques. As described above, the module 20 can be used to illuminate a person's skin and measure changes in light absorption. If the module 20 is attached without compressing the skin, a pressure pulse can also be seen from the venous plexus, as a small secondary peak. The change in volume caused by the pressure pulse is detected by illuminating the skin with the light from a LED or other light source 24 and then measuring the amount of light reflected to a photodiode 34A. Each cardiac cycle appears as a peak. Because blood flow to the skin can be modulated by other physiological systems, the PPG also can be used to monitor breathing, hypovolemia, and other circulatory conditions.

The foregoing paragraphs illustrate particular examples of how the integrated bio-sensor module 20 can be used, depending on the particular implementation. The module 20 also may be configured to measure other physiological conditions of a living being.

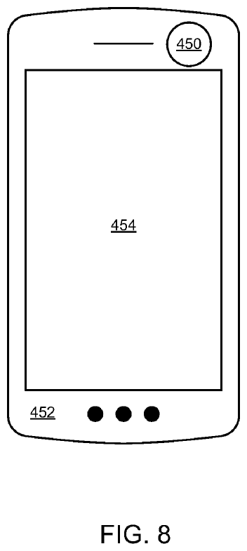

As shown in FIG. 8, a biosensor system 450 including an integrated biosensor module 20 as described above can be incorporated into a portable (e.g., handheld) or other host computing device 452, such as a smartphone (as shown), a computer tablet, a wearable computing device, a smart health device, or a smart patch device. In such implementations, the biosensor module 20 can be disposed, for example, under a cover glass of the host computing device. In some cases, the biosensor system can be used for automotive applications. The sensor system 450 can be operable by a user, e.g., under control of an application executing on the computing device 452, to conduct physiological measurements such as those described above. A test result can be displayed on a display screen 454 of the computing device 452, e.g., to provide substantially immediate feedback to the user about the measured physiological data.

Various modifications will be readily apparent and can be made to the foregoing examples. Features described in connection with different embodiments may be incorporated into the same implementation in some cases, and various features described in connection with the foregoing examples may be omitted from some implementations. Thus, other implementations are within the scope of the claims.

1. An integrated optical biosensor module comprising:
one or more light sources operable to produce light for emission from the module;
an integrated circuit chip including a photosensitive region, the photosensitive region including one or more photodetectors operable to detect light produced by the one or more light sources and reflected by a subject that is outside the module, wherein the integrated circuit chip is operable to determine a physiological condition of the subject based on signals from the one or more photodetectors;
a clear mold covering encapsulating the one or more light sources, wherein the clear mold covering includes one or beam shaping elements each of which is disposed so as to intersect a path of a light beam from an associated one of the one or more light source.

2. The module of claim 1 wherein each of the one or more beam shaping elements is a molded lens composed of a same material as the clear mold covering.

3. The module of claim 2 wherein the clear mold covering and the one or more beam shaping elements are composed of an epoxy resin.

4. The module of claim 2 including a plurality of light sources, wherein the clear mold covering includes a plurality of beam shaping elements each of which is disposed so as to intersect a path of a light beam from a respective one of the light sources.

5. The module of claim 4 wherein at least one of the beam shaping elements is asymmetric with respect to an optical axis of the light beam produced by the respective light source.

6. The module of claim 4 wherein each of the respective beam shaping elements is operable to direct a light beam from a respective one of the light sources in a respective direction that differs from a direction in which a light beam from at least another one of the light sources is directed by a different one of the beam shaping elements.

7. The module of claim 4 wherein each of the light sources is operable to produce light of a different wavelength, the module including a plurality of photodetectors each of which is operable to detect light produced by a respective one of the light sources and reflected by the subject.

8. The module of claim 4 wherein at least one of the light sources is operable to produce infra-red light.

9. The module of claim 4 wherein at least one of the light sources is operable to produce visible light.

10. The module of claim 1 wherein the clear mold covering is transparent to the light produced by the one or more light sources.

11. The module of claim 1 further including a housing defining an interior region in which the one or more light sources and the integrated circuit chip are disposed.

12. The module of claim 11 wherein the housing has a first aperture over the clear mold covering.

13. The module of claim 12 further including a second aperture over the integrated circuit chip.

14. The module of claim 1 wherein the integrated circuit chip is operable to determine an oxygen saturation level of the subject based on the signals from the one or more photodetectors.

15. The module of claim 1 wherein the integrated circuit chip is operable to determine an pulse rate of the subject based on the signals from the one or more photodetectors.

16. The module of claim 1 wherein the integrated circuit chip is operable to determine a heart rate of the subject based on the signals from the one or more photodetectors.

17. A host computing device comprising:
a cover glass;
a module according to claim 1 disposed adjacent the cover glass;
an application executable on the host computing device and operable to cause the module to perform a physiological measurement on the subject based on light produced by the one or more light sources, reflected by the subject, and sensed by the one or more photodetectors; and
a display screen operable to display data indicative of the physiological condition of the subject based on the signals from the one or more photodetectors.

18. A system comprising:
an integrated optical biosensor module comprising:
one or more light sources operable to produce light for emission from the module;
an integrated circuit chip including a photosensitive region, the photosensitive region including one or more photodetectors operable to detect light produced by the one or more light sources and reflected by a subject that is outside the module; and
a clear mold covering encapsulating the one or more light sources, wherein the clear mold covering includes one or beam shaping elements each of which is disposed so as to intersect a path of a light beam from an associated one of the one or more light sources;
the system further including a processor coupled to the integrated circuit chip and operable to determine a physiological condition of the subject based on signals from the one or more photodetectors.
